(19) United States
(12) Patent Application Publication
(10) Pub. No.: US 2025/0304610 A1
(43) Pub. Date: Oct. 2, 2025

(54) TREHALOSE-BASED SURFACTANTS

(71) Applicant: MEDXCELL SA, Montreux (CH)

(72) Inventors: Solene PASSEMARD, Lunel (FR); Sandrine GERBER, Etoy (CH); Francois NOVERRAZ, Lausanne (CH); Baptiste ROBIN, Montpellier (FR)

(21) Appl. No.: 18/865,047

(22) PCT Filed: May 16, 2023
(86) PCT No.: PCT/EP2023/063068

§ 371 Date: Nov. 12, 2024

(30) Foreign Application Priority Data
May 17, 2022 (EP) 22173849.5

(51) Int. Cl. C07H 13/06; A61K 39/00; A61K 39/215; A61K 47/26

(52) U.S. Cl. CPC C07H 13/06; A61K 39/215; A61K 47/26; A61K 2039/543; A61K 2039/544

(57) ABSTRACT

Provided herein are compounds that function as surfactants and/or excipients, methods of producing such compounds, methods of using such compounds and compositions comprising such compounds preferably in combination with one or more additional agents or therapies.

### FIELD OF THE INVENTION

Provided herein are compounds that function as surfactants and/or excipients, methods of producing such compounds, methods of using such compounds and compositions comprising such compounds preferably in combination with one or more additional agents or therapies.

### BACKGROUND OF THE INVENTION

Excipients are large class of molecule used in the pharmaceutical formulation of chemical drugs, antibodies and proteins. They are also involved in the improvement of shelf-life stability of biomolecules during storage, or for biomolecule cryopreservation and freeze-drying.

Excipients used in the pharmaceutical formulation of antibodies and proteins can be classified as:
Bulking agents e.g. the polyol/disaccharide/polysaccharides family as well as natural polysaccharides and amino acids,
Tonicity agents based on sugar materials,
Buffering agents such as organic and inorganic salts,
Surfactants, to reduce surface tension while improving drugs solubility,
Antioxidants,
Chelators,
Preservatives.

Excipients are important for biomaterials preservation steps in research and development industries. Biomolecules are usually cryopreserved at −80° C. or alternatively freeze-dried. Maintaining the materials cryopreserved is a huge cost in terms of electricity and nitrogen. Also freeze-drying is less costly, an important loss of active materials is associated. It is challenging to keep the materials intact when the materials composition (loss of water), temperature and pressure have been impaired. Macromolecules can undergo chemical instabilities under stress conditions (changes of temperature, exposure to light, oxygen, or chemical and shear stress) leading to a loss of biological activity. The instability can refer to the irreversible denaturation (the protein loss its tertiary or its secondary structure) and aggregation (protein self-assemble in an irreversible way).

Among surfactants, polysorbates PS20 and PS80 are widely approved for parenteral administration of antibodies. However, it has been reported that they impair long-term storage due to the presence of peroxides residuals from the poly(ethylene glycol) entities. This causes protein oxidation and enhance protein degradation. Moreover, it has been shown that polysorbates damage cell membrane integrity and impair mitochondrial function at a concentration of 1 to 2 v/v % on BEAES2B bronchial epithelial cell.

Thus, better and safer excipients must be developed for the stability, long-term storage of macromolecules as well as for administration of macromolecules if needed.

### BRIEF DESCRIPTION OF THE FIGURES

FIG. 1: Aggregation monitored overtime by DLS during storage at 4° C. The graph shows the total area under curve (AUC) of the peaks corresponding to antibody aggregates for antibody 1 mg/mL in different stabilizing media, after 0, 5 and 4 days of storage

FIG. 2: Nebulization-triggered aggregation assessed by DLS. The graph shows the total area under curve (AUC) of the peaks corresponding to antibody aggregates before (white columns) and after (striped columns) nebulization, for antibody at a concentration of 1 mg/mL in each stabilization medium.

FIG. 3: Nebulization-triggered aggregation assessed by scattering measurement module of Nano-DSF. The graph represents the difference of sample scattering at 70° C. and at 20° C. before (white columns) and after (striped columns) nebulization, for antibody at a concentration of 1 mg/mL in each stabilization medium.

FIG. 4: Aggregation assessed by DLS after freeze-drying. The graph shows the total area under curve (AUC) of the peaks corresponding to antibody aggregates for antibody at a concentration of 1 mg/mL in each stabilization medium.

FIG. 5: Graphical representation of the proportion of SARS-COV-2 spike-expressing HEK293 cells marked by AF488-conjugated anti-Fc IgG, measured by FACS. Marked cells are supposed to be bound to the antibody of interest in different stabilizing media.

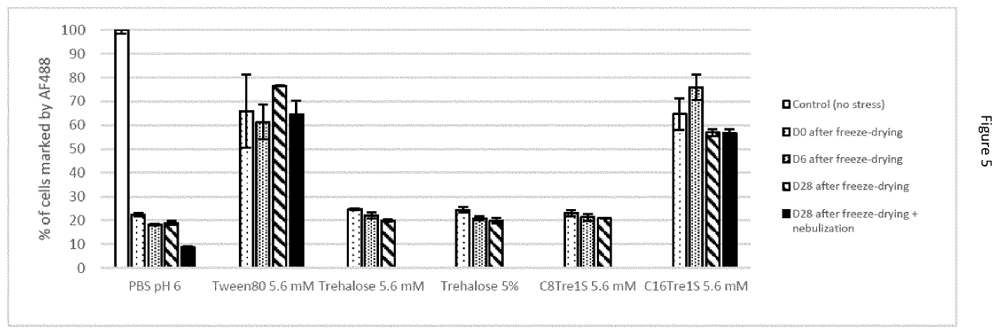

FIG. 6: Cytotoxicity of trehalose excipients and polysorbate 80 on NIH-3T3 cells after 48 h, measured by colorimetric dosage after MTS staining.

### SUMMARY OF THE INVENTION

The present invention provides a compound of formula I

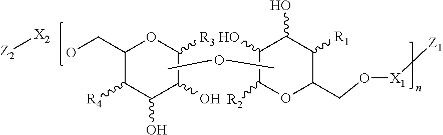

or a salt, stereoisomer, polymorph or mixture of stereoisomers thereof,
wherein
R₁ is selected from-OH, an alkyl, optionally substituted with OH, or is linked to R₃ via an oxygen;
R₂ is selected from-OH, an alkyl, optionally substituted with OH, or is linked to R₃ via an oxygen;
R₃ is selected from-OH, an alkyl, optionally substituted with OH, or is linked to R₁ or R₂ via an oxygen;
R₄ is selected from-OH or an alkyl, optionally substituted with-OH;
X₁ is independently selected from —(C═O), and —(C—O)-A-(C═O), wherein A is an alkyl, an alkenyl, an alkynyl or PEG;
X₂ is independently selected from H, or —(C═O);
Z₁ is —OH;
and,
   when X₂ is C═O, then Z₂ is independently selected from alkyl, alkenyl, alkynyl, PEG, succinyl, —NH alkyl, —NH alkenyl, —NH alkynyl, NH-PEG, —S alkyl, —S alkynyl, —O alkyl, —O alkenyl, and —O alkynyl
   when X₂ is H, then Z₂ is absent;
and n is an integer between 1-20.

The present invention also provides a composition comprising a compound of formula I as described herein.

The present invention further provides a pharmaceutical composition comprising a compound of formula I as described herein.

The present invention further provides a method for preparing a compound of formula I, the method comprising
a sequential and selective functionalization of the two primary alcohols of trehalose while maintaining the other hydroxyl moieties unreacted,
allowing the conjugation first to a fatty acid chain and then to a carboxylated side chain wherein the carboxylic functionality is introduced in the form of a benzylic ester which is removed at the last step of the procedure.

The present invention further provides a method for preparing a polymer of a compound of formula I, the method comprising autocondensating an activated trehalose derivative in the presence of a coupling agent.

### DETAILED DESCRIPTION

As used in the present disclosure, the following words and phrases are generally intended to have the meanings as set forth below unless expressly indicated otherwise or the context in which they are used indicates otherwise.

The following description sets forth exemplary methods, parameters and the like. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

A dash (“-”) that is not between two letters or symbols is used to indicate a point of attachment for a substituent. For example, —CH₂ is attached through the carbon atom. A dash at the front or end of a chemical group is a matter of convenience; chemical groups may be depicted with or without one or more dashes without losing their ordinary meaning. Unless chemically or structurally required, no directionality is indicated or implied by the order in which a chemical group is written or named.

The prefix “Cᵤ₋ᵥ” indicates that the following group has from u to v carbon atoms. For example, “C₁₋₆ alkyl” indicates that the alkyl group has from 1 to 6 carbon atoms.

Reference to “about” a value or parameter herein includes (and describes) embodiments that are directed to that value or parameter per se. In certain embodiments, the term “about” includes the indicated amount±10%. In other embodiments, the term “about” includes the indicated amount±5%. In certain other embodiments, the term “about” includes the indicated amount±1%. Also, to the term “about X” includes description of “X”. Also, the singular forms “a” and “the “include plural references unless the context clearly dictates otherwise. Thus, e.g., reference to “the compound” includes a plurality of such compounds and reference to “the assay” includes reference to one or more assays and equivalents thereof known to those skilled in the art.

The term “substituted” means that any one or more hydrogen atoms on the designated atom or group is replaced with one or more substituents other than hydrogen, provided that the designated atom's normal valence is not exceeded. The one or more substituents include, but are not limited to, alkyl, alkenyl, alkynyl, alkoxy, acyl, amino, amido, amidino, aryl, azido, carbamoyl, carboxyl, carboxyl ester, cyano, guanidino, halo, haloalkyl, heteroalkyl, heteroaryl, heterocycloalkyl, hydroxy, hydrazino, imino, oxo, nitro, alkylsulfinyl, sulfonic acid, alkylsulfonyl, thiocyanate, thiol, thione, or combinations thereof. Polymers or similar indefinite structures arrived at by defining substituents with further substituents appended ad infinitum (e.g., a substituted aryl having a substituted alkyl which is itself substituted with a substituted aryl group, which is further substituted by a substituted heteroalkyl group, etc.) are not intended for inclusion herein. Unless otherwise noted, the maximum number of serial substitutions in compounds described herein is three. For example, serial substitutions of substituted aryl groups with two other substituted aryl groups are limited to ((substituted aryl)substituted aryl) substituted aryl. Similarly, the above definitions are not intended to include impermissible substitution patterns (e.g., methyl substituted with 5 fluorines or heteroaryl groups having two adjacent oxygen ring atoms). Such impermissible substitution patterns are well known to the skilled artisan. When used to modify a chemical group, the term “substituted” may describe other chemical groups defined herein. For example, the term “substituted aryl” includes, but is not limited to, “alkylaryl.” Unless specified otherwise, where a group is described as optionally substituted, any substituents of the group are themselves unsubstituted.

A “substituted” group also includes embodiments in which a monoradical substituent is bound to a single atom of the substituted group (e.g., forming a branch), and also includes embodiments in which the substituent may be a diradical bridging group bound to two adjacent atoms of the substituted group, thereby forming a fused ring on the substituted group.

“Alkyl” refers to an unbranched or branched saturated hydrocarbon chain. As used herein, alkyl has 1 to 20 carbon atoms (i.e., C₁₋₂₀ alkyl), 1 to 8 carbon atoms (i.e., C₁₋₈ alkyl), 1 to 6 carbon atoms (i.e., C₁₋₆ alkyl), or 1 to 4 carbon atoms (i.e., C₁₋₄ alkyl). Examples of alkyl groups include methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e., —(CH₂)₃CH₃), sec-butyl (i.e., —CH(CH₃)CH₂CH₃), isobutyl (i.e., —CH₂CH(CH₃)₂) and tert-butyl (i.e., —C(CH₃)₃); and “propyl” includes n-propyl (i.e., —(CH₂)₂CH₃) and isopropyl (i.e., —CH(CH₃)₂).

“Alkenyl” refers to an aliphatic group containing at least one carbon-carbon double bond and having from 2 to 20 carbon atoms (i.e., C₂₋₂₀ alkenyl), 2 to 8 carbon atoms (i.e., C₂₋₈ alkenyl), 2 to 6 carbon atoms (i.e., C₂₋₆ alkenyl), or 2 to 4 carbon atoms (i.e., C₂₋₄ alkenyl). Examples of alkenyl groups include ethenyl, propenyl, butadienyl (including 1,2-butadienyl, and 1,3-butadienyl).

“Alkynyl” refers to an aliphatic group containing at least one carbon-carbon triple bond and having from 2 to 20 carbon atoms (i.e., C₂₋₂₀ alkynyl), 2 to 8 carbon atoms (i.e., C₂₋₈ alkynyl), 2 to 6 carbon atoms (i.e., C₂₋₆ alkynyl), or 2 to 4 carbon atoms (i.e., C₂₋₄ alkynyl). The term “alkynyl” also includes those groups having one triple bond and one double bond.

“Alkoxy” refers to the group “alkyl-O-” or “—O-alkyl”. Examples of alkoxy groups include methoxy, ethoxy, n-propoxy, iso-propoxy, n-butoxy, tert-butoxy, sec-butoxy, n-pentoxy, n-hexoxy, and 1,2-dimethylbutoxy.

“Amino” refers to the group —NR^{y}R^{z} wherein R^{y} and R^{z} are independently selected from hydrogen, alkyl, haloalkyl, cycloalkyl, aryl, heterocyclyl, or heteroaryl; each of which may be optionally substituted.

The term “alkylsulfinyl” refers to the group —SO-alkyl, where alkyl is as defined above, and includes optionally substituted alkyl groups as also defined above.

“Cycloalkyl” refers to a saturated or partially saturated cyclic alkyl group having a single ring or multiple rings including fused, bridged, and spiro ring systems. As used herein, cycloalkyl has from 3 to 20 ring carbon atoms (i.e., C₃₋₂₀ cycloalkyl), 3 to 12 ring carbon atoms (i.e., C₃₋₁₂ cycloalkyl), 3 to 10 ring carbon atoms (i.e., C₃₋₁₀ cycloalkyl), 3 to 8 ring carbon atoms (i.e., C₃₋₈ cycloalkyl), or 3 to 6 ring carbon atoms (i.e., C₃₋₆ cycloalkyl). Examples of cycloalkyl groups include cyclopropyl, cyclobutyl, cyclopentyl, and cyclohexyl.

As used herein the term “cycloalkenyl” groups means the non-aromatic carbocyclic group having at least one double bond.

“Cyanoalkyl” refers to an alkyl group substituted with cyano (CN).

“Halogen” or “halo” includes fluoro, chloro, bromo, and iodo.

The term “haloalkyl” refers to a monoradical or diradical having the indicated carbon atoms of the alkyl group wherein one or more hydrogen atoms have been substituted by a halogen. Examples of haloalkyl groups include —CH₂F, —CHF₂, —CF₃, —CH₂CF₃, —CHFCH₂F, —CF₂—, —CHF—, and the like. Similarly, the term “haloalkoxy”, e.g., —O—C₁₋₃haloalkyl, refers to an alkoxy group wherein one or more hydrogen atoms of the alkyl group have been substituted by a halogen. Examples of haloalkoxy groups include —OCH₂F, —OCHF₂, —OCF₃, —OCH₂CF₃, —OCHFCH₂F, and the like. One of skill in the art is aware that similar definitions apply for the alkenyl and alkynyl analogs (e.g., C₂₋₄haloalkenyl, —O—C₂₋₄haloalkynyl) of the above.

“Heteroalkyl” refers to an alkyl group in which one or more of the carbon atoms (and any associated hydrogen atoms) are each independently replaced with the same or different heteroatomic group. The term “heteroalkyl” includes unbranched or branched saturated chain having carbon and heteroatoms. By way of example, 1, 2 or 3 carbon atoms may be independently replaced with the same or different heteroatomic group. Heteroatomic groups include, but are not limited to, —NR—, —O—, —S—, —SO—, —SO₂—, and the like, where R is H, alkyl, aryl, cycloalkyl, heteroalkyl, heteroaryl, or heterocycloalkyl, each of which may be optionally substituted. Examples of heteroalkyl groups include —OCH₃, —CH₂OCH₃, —SCH₃, —CH₂SCH₃, —NRCH₃, and —CH₂NRCH₃, where R is hydrogen, alkyl, aryl, arylalkyl, heteroalkyl, or heteroaryl, each of which may be optionally substituted. As used herein, heteroalkyl includes 1 to 10 carbon atoms, 1 to 8 carbon atoms, or 1 to 4 carbon atoms; and 1 to 3 heteroatoms, 1 to 2 heteroatoms, or 1 heteroatom.

“Heterocycloalkyl” refers to a saturated or unsaturated cyclic alkyl group, with one or more ring heteroatoms independently selected from nitrogen, oxygen and sulfur. A heterocycloalkyl may be a single ring or multiple rings wherein the multiple rings may be fused, bridged, or spiro. As used herein, heterocycloalkyl has 2 to 20 ring carbon atoms (i.e., C₂₋₂₀ heterocycloalkyl), 2 to 12 ring carbon atoms (i.e., C₂₋₁₂ heterocycloalkyl), 2 to 10 ring carbon atoms (i.e., C₂₋₁₀ heterocycloalkyl), 2 to 8 ring carbon atoms (i.e., C₂₋₈ heterocycloalkyl), 3 to 12 ring carbon atoms (i.e., C₃₋₁₂ heterocycloalkyl), 3 to 8 ring carbon atoms (i.e., C₃₋₈ heterocycloalkyl), or 3 to 6 ring carbon atoms (i.e., C₃₋₆ heterocycloalkyl); having 1 to 5 ring heteroatoms, 1 to 4 ring heteroatoms, 1 to 3 ring heteroatoms, 1 to 2 ring heteroatoms, or 1 ring heteroatom independently selected from nitrogen, sulfur or oxygen. Examples of heterocycloalkyl groups include pyrrolidinyl, piperidinyl, piperazinyl, oxetanyl, dioxolanyl, azetidinyl, and morpholinyl. As used herein, the term “bridged-heterocycloalkyl” refers to a four- to ten-membered cyclic moiety connected at two non-adjacent atoms of the heterocycloalkyl with one or more (e.g., 1 or 2) four- to ten-membered cyclic moiety having at least one heteroatom where each heteroatom is independently selected from nitrogen, oxygen, and sulfur. As used herein, bridged-heterocycloalkyl includes bicyclic and tricyclic ring systems. Also used herein, the term “spiro-heterocycloalkyl” refers to a ring system in which a three- to ten-membered heterocycloalkyl has one or more additional ring, wherein the one or more additional ring is three- to ten-membered cycloalkyl or three- to ten-membered heterocycloalkyl, where a single atom of the one or more additional ring is also an atom of the three- to ten-membered heterocycloalkyl. Examples of spiro-heterocycloalkyl include bicyclic and tricyclic ring systems, such as 2-oxa-7-azaspiro[3.5]nonanyl, 2-oxa-6-azaspiro[3.4]octanyl, and 6-oxa-1-azaspiro[3.3]heptanyl.

“Acyl” refers to a group —C(═O)R, wherein R is hydrogen, alkyl, cycloalkyl, heterocycloalkyl, aryl, heteroalkyl, or heteroaryl; each of which may be optionally substituted, as defined herein. Examples of acyl include formyl, acetyl, cyclohexylcarbonyl, cyclohexylmethyl-carbonyl, and benzoyl.

The term “N-alkylated” means an alkyl group is substituted for one of the hydrogen atoms of a mono substituted amine, or a di-substituted amine group or a tri substituted amine group. When the alkylation is on a tri-substituted amine group an alkonium salt is generated i.e., a positive charge is generated on the nitrogen atom. N-alkylation is commonly associated with alkyl substitution on a ring nitrogen atom.

The term “oxo” refers to a group ═O.

The term “carboxy” refers to a group —C(O)—OH.

The term “ester” or “carboxyl ester” refers to the group —C(O)OR, where R is alkyl, cycloalkyl, aryl, heteroaryl, or heterocyclyl, which may be optionally further substituted, for example, by alkyl, alkoxy, halogen, CF₃, amino, substituted amino, cyano or —SOₙR^{f}, in which R^{f}is alkyl, aryl, or heteroaryl, and n is 0, 1 or 2.

The term “substituted amino” refers to the group —NRR, where each R is independently hydrogen, alkyl, cycloalkyl, aryl, heteroaryl, or heterocyclyl, each of which may be optionally substituted, or a group as described or exemplified herein, or where both R groups are joined to form a heterocyclic group (e.g., morpholino) as described or exemplified herein, which also may be optionally substituted.

The term “amido” refers to the group —C(O)NRR where each R is independently hydrogen, alkyl, cycloalkyl, aryl, heteroaryl, or heterocyclyl, each of which may be optionally substituted, or a group as described or exemplified herein, or where both R groups are joined to form a heterocyclic group (e.g., morpholino) as described or exemplified herein, which also may be optionally substituted.

The term “sulfoxide” refers to a group —SOR, in which R is alkyl, cycloalkyl, heterocyclyl, aryl, or heteroaryl, each of which may be optionally substituted.

As used herein, the terms “alkylcycloalkyl,” “alkylaryl,” “alkylheteroaryl” and “alkylheterocyclyl” are intended to refer to a cycloalkyl, aryl, heteroaryl or heterocyclyl group which is bound to the remainder of the molecule via an alkyl moiety, where the terms “alkyl,” “cycloalkyl,” “aryl,” “heteroaryl” and “heterocyclyl” are as defined herein.

Exemplary alkylaryl groups include benzyl, phenethyl, and the like.

“Optional” or “optionally” means that the subsequently described event or circumstance may or may not occur, and that the description includes instances where said event or circumstance occurs and instances in which it does not.

As used herein, polyethylene glycol or “PEG” refers to a polymer expressed as H—(O—CH2-CH2)n-OH, where n represents the number of times the O—CH2-CH2 (oxyethylene) moiety is repeated; n can range widely, because PEG comes in a wide variety of molecular weights. For instance, n can be about 33 for lower-molecular weight polyethylene glycols (˜1500 g/mol), ranging up to about 227 for higher molecular weight polyethylene glycols (˜10,000 g/mol) such as about 454 for ˜20,000 g/mol molecular-weight PEG; and 908 for ˜40,000 molecular-weight PEG; and even higher for higher-molecular-weight PEG varieties.

Certain commonly used alternative chemical names may be used. For example, a divalent group such as a divalent “alkyl” group, a divalent “aryl” group, etc., may also be referred to as an “alkylene” group or an “alkylenyl” group, an “arylene” group or an “arylenyl” group, respectively. Also, unless indicated explicitly otherwise, where combinations of groups are referred to herein as one moiety, e.g., arylalkyl, the last mentioned group contains the atom by which the moiety is attached to the rest of the molecule.

Where a given group (moiety) is described herein as being attached to a second group and the site of attachment is not explicit, the given group may be attached at any available site of the given group or to any available site of the second group. For example, an “alkyl-substituted phenyl”, where the attachment sites are not explicit, may have any available site of the alkyl group attached to any available site of the phenyl group. In this regard, an “available site” is a site of the group at which hydrogen of the group may be replaced with a substituent.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. Also not included are infinite numbers of substituents, whether the substituents are the same or different. In such cases, the maximum number of such substituents is three. Each of the above definitions is thus constrained by a limitation that, for example, substituted aryl groups are limited to -substituted aryl-(substituted aryl)-substituted aryl.

“Isomers” are different compounds that have the same molecular formula. Isomers include stereoisomers, enantiomers and diastereomers.

“Stereoisomers” are isomers that differ only in the way the atoms are arranged in space.

“Enantiomers” are a pair of stereoisomers that are non-superimposable mirror images of each other. A 1:1 mixture of a pair of enantiomers is a “racemic” mixture. The term “(I)” is used to designate a racemic mixture where appropriate.

“Diastereoisomers” are stereoisomers that have at least two asymmetric atoms, but which are not mirror-images of each other.

The compounds of the disclosure may possess one or more asymmetric centers and may be produced as a racemic mixture or as individual enantiomers or diastereoisomers. The number of stereoisomers present in any given compound of a given formula depends upon the number of asymmetric centers present (there are 2” stereoisomers possible where n is the number of asymmetric centers). The individual stereoisomers may be obtained by resolving a racemic or non-racemic mixture of an intermediate at some appropriate stage of the synthesis or by resolution of the compound by conventional means. The individual stereoisomers (including individual enantiomers and diastereoisomers) as well as racemic and non-racemic mixture of stereoisomers are encompassed within the scope of the present disclosure, all of which are intended to be depicted by the structures of this specification unless otherwise specifically indicated.

The absolute stereochemistry is specified according to the Cahn Ingold Prelog R S system. When the compound is a pure enantiomer the stereochemistry at each chiral carbon may be specified by either R or S. A resolved compound whose absolute configuration is unknown may be designated (+) or (−) depending on the direction (dextro- or laevorotary) that it rotates the plane of polarized light at the wavelength of the sodium D line.

“Trehalose” as used herein refers to a nonreducing disaccharide consisting of two glucose units. It is found in many plants, microorganisms, and animals and is involved in glucose storage, signalling and regulation, structural and transport role, membranes and proteins protectant and stabilizers. It is widely approved in biomolecules formulations for pharmaceutical development. Trehalose stabilizes biomolecules by its ability to form hydrogen bonds with the amino acids present on their surface.

The term “polymorph” refers to different crystal structures of a crystalline compound.

The different polymorphs may result from differences in crystal packing (packing polymorphism) or differences in packing between different conformers of the same molecule (conformational polymorphism).

The term “solvate” refers to a complex formed by combining a compound of formula (I), or any other formula as disclosed herein and a solvent.

The term “hydrate” refers to the complex formed by the combining of a compound of formula (I), or any formula disclosed herein, and water.

The term “polymorph”, as used herein, refers to different crystalline forms of the same compound and other solid state molecular forms including pseudo-polymorphs, such as hydrates (e.g., bound water present in the crystalline structure as discussed above) and solvates (e.g., bound solvents other than water) of the same compound. Different crystalline polymorphs have different crystal structures due to a different packing of the molecules in the lattice. This results in a different crystal symmetry and/or unit cell parameters which directly influences its physical properties such the X-ray diffraction characteristics of crystals or powders. A different polymorph, for example, will in general diffract at a different set of angles and will give different values for the intensities. Therefore, X-ray powder diffraction can be used to identify different polymorphs, or a solid form that comprises more than one polymorph, in a reproducible and reliable way (S. Byrn et al, Pharmaceutical Solids: A Strategic Approach to Regulatory Considerations, Pharmaceutical research, Vol. 12, No. 7, p. 945-954, 1995; J. K. Haleblian and W. McCrone, Pharmacetical Applications of Polymorphism, Journal of Pharmaceutical Sciences, Vol. 58, No. 8, p. 911-929, 1969).

The term “prodrug” refers to compounds of formula (I), or derivatives of formula (I) disclosed herein, that include chemical groups which, in vivo, can be converted and/or can be split off from the remainder of the molecule to provide for the active drug. Pharmaceutically acceptable salts or biologically active metabolites thereof of the prodrug of a compound of formula (I) are also within the ambit of the present disclosure.

Any formula or structure given herein, including formula (I), or any formula disclosed herein, is intended to represent unlabeled forms as well as isotopically labeled forms of the compounds. Isotopically labeled compounds have structures depicted by the formulas given herein except that one or more atoms are replaced by an isotope having a selected atomic mass or mass number. Examples of isotopes that can be incorporated into compounds of the disclosure include isotopes of hydrogen, carbon, nitrogen, oxygen, phosphorus, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl, and ¹²⁵I. Various isotopically labeled compounds of the present disclosure, for example those into which radioactive isotopes such as ³H, ¹³C and ¹⁴C are incorporated, are within the ambit of the present disclosure. Such isotopically labelled compounds may be useful in metabolic studies, reaction kinetic studies, detection or imaging techniques, such as positron emission tomography (PET) or single-photon emission computed tomography (SPECT) including drug or substrate tissue distribution assays or in treatment of patients. Such isotopically labeled analogs of compounds of the present disclosure may also be useful for treatment of diseases disclosed herein because they may provide improved pharmacokinetic and/or pharmacodynamic properties over the unlabeled forms of the same compounds. Such isotopically leveled forms of or analogs of compounds herein are within the ambit of the present disclosure. One of skill in the art is able to prepare and use such isotopically labeled forms following procedures for isotopically labeling compounds or aspects of compounds to arrive at isotopic or radiolabeled analogs of compounds disclosed herein.

The term “pharmaceutically acceptable salt” of a given compound refers to salts that retain the biological effectiveness and properties of the given compound, and which are not biologically or otherwise undesirable. Pharmaceutically acceptable base addition salts can be prepared from inorganic and organic bases. Salts derived from inorganic bases include, by way of example only, sodium, potassium, lithium, ammonium, calcium and magnesium salts. Salts derived from organic bases include, but are not limited to, salts of primary, secondary and tertiary amines, such as alkyl amines, dialkyl amines, trialkyl amines, substituted alkyl amines, di(substituted alkyl) amines, tri(substituted alkyl) amines, alkenyl amines, dialkenyl amines, trialkenyl amines, substituted alkenyl amines, di(substituted alkenyl) amines, tri(substituted alkenyl) amines, cycloalkyl amines, di(cycloalkyl) amines, tri(cycloalkyl) amines, substituted cycloalkyl amines, di-substituted cycloalkyl amine, tri-substituted cycloalkyl amines, cycloalkenyl amines, di(cycloalkenyl) amines, tri(cycloalkenyl) amines, substituted cycloalkenyl amines, di-substituted cycloalkenyl amine, tri-substituted cycloalkenyl amines, aryl amines, diaryl amines, triaryl amines, heteroaryl amines, diheteroaryl amines, triheteroaryl amines, heterocyclic amines, diheterocyclic amines, triheterocyclic amines, mixed di- and tri-amines where at least two of the substituents on the amine are different and are selected from alkyl, substituted alkyl, alkenyl, substituted alkenyl, cycloalkyl, substituted cycloalkyl, cycloalkenyl, substituted cycloalkenyl, aryl, heteroaryl, heterocyclic, and the like. Also included are amines where the two or three substituents, together with the amino nitrogen, form a heterocyclic or heteroaryl group. Amines are of general structure N(R³⁰)(R³¹)(R³²), wherein mono-substituted amines have two of the three substituents on nitrogen (R³⁰, R³¹, and R³²) as hydrogen, di-substituted amines have one of the three substituents on nitrogen (R³⁰, R³¹, and R³²) as hydrogen, whereas tri-substituted amines have none of the three substituents on nitrogen (R³⁰, R³¹, and R³²) as hydrogen. R³⁰, R³¹, and R³² are selected from a variety of substituents such as hydrogen, optionally substituted alkyl, aryl, heteroaryl, cycloalkyl, cycloalkenyl, heterocyclyl, and the like.

Specific examples of suitable amines include, by way of example only, isopropyl amine, trimethyl amine, diethyl amine, tri(iso-propyl) amine, tri(n-propyl) amine, ethanolamine, diethanolamine, 2-dimethylamino ethanol, lysine, arginine, histidine, caffeine, procaine, hydrabamine, choline, betaine, ethylenediamine, glucosamine, N-alkylglucamines, theobromine, purines, piperazine, piperidine, morpholine, N-ethylpiperidine, and the like.

Pharmaceutically acceptable acid addition salts may be prepared from inorganic and organic acids. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like.

Sugar-based surfactants with diverse structures can be a good alternative to polysorbates surfactants. There are mainly produced by enzymatic synthesis from microorganisms. They showed low toxicity, biodegradability and biocompatibility and are produced from cheaper and renewable energy sources. Pharmaceutical, food, cosmetic, textile, oil, and agricultural applications manifest interest in using biosurfactants for their benefits. However, the uses in pharmaceutical industries are limited due to the risk of endotoxin contamination of biosurfactants made from bacteria. Their production leads to glycolipids mixture with different properties, leading to inconsistent and confusing results. More recently, sugar-based surfactants have been synthetically made. Among them, trehalose lipids are constituted from trehalose attached through an ester bond to lipids at various positions.

Provided herein are compounds that function as surfactants and/or excipients, methods of producing such compounds, methods of using such compounds and compositions comprising such compounds preferably in combination with one or more additional agents or therapies.

It is further contemplated that all aspects directed to compounds include any pharmaceutically acceptable salt, stereoisomer, polymorph, mixture of stereoisomers, solvate, or a prodrug thereof.

The present disclosure provides, in some aspects, a compound of formula (I)

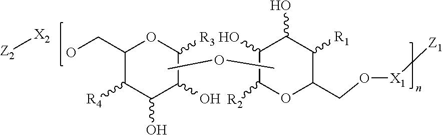

or a salt, stereoisomer, polymorph or mixture of stereoisomers thereof,
wherein
R₁ is selected from —OH, an alkyl, optionally substituted with OH, or is linked to R₃ via an oxygen;
R₂ is selected from —OH, an alkyl, optionally substituted with OH, or is linked to R₃ via an oxygen;
R₃ is selected from —OH, an alkyl, optionally substituted with OH, or is linked to R₁ or R₂ via an oxygen;
R₄is selected from —OH or an alkyl, optionally substituted with —OH;
X₁ is independently selected from —(C═O) and —(C═O)-A-(C═O), wherein A is an alkyl, an alkenyl, an alkynyl or PEG;
X₂ is independently selected from H or —(C═O);
Z₁ is —OH;
and,
   when X₂ is C═O, then Z₂ is independently selected from alkyl, alkenyl, alkynyl, PEG, succinyl, —NH alkyl, —NH alkenyl, —NH alkynyl, NH-PEG, —S alkyl, —S alkynyl, —O alkyl, —O alkenyl, and —O alkynyl
   when X₂ is H, then Z₂ is absent;
and n is an integer between 1-20.

In one aspect, R₁ is —OH.

In one aspect, R₂ is linked to R₃ via an oxygen.

In one aspect, R₄ is OH.

In one aspect, X₁ is —(C═O)—CH2-CH2-(C═O).

In one aspect, Z₁ is —OH.

In one aspect, X₂ is —(C═O).

In one aspect, Z₂ is an alkyl, alkenyl or is absent. Examples of alkyls include, but are not limited to, an alkyl having 1 to 20 carbon atoms (i.e., C1-20 alkyl), 1 to 8 carbon atoms (i.e., C1-8 alkyl), 1 to 6 In one aspect, carbon atoms (i.e., C1-6 alkyl), or 1 to 4 carbon atoms (i.e., C1-4 alkyl) as described above. Examples of alkenyls include, but are not limited to alkenyl having 2 to 20 carbon atoms (i.e., C2-20 alkenyl), 2 to 8 carbon atoms (i.e., C2-8 alkenyl), 2 to 6 carbon atoms (i.e., C2-6 alkenyl), or 2 to 4 carbon atoms (i.e., C2-4 alkenyl). Examples of alkenyl groups include ethenyl, propenyl, butadienyl (including 1,2-butadienyl, and 1,3-butadienyl).

In one aspect, the compound of formula (I) is selected from the group consisting of

| | |
|---|---|
| 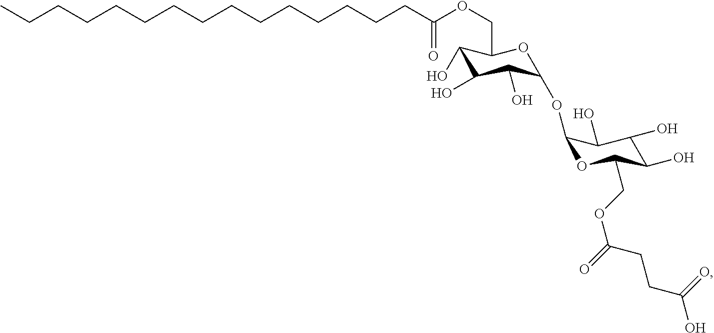 | (Compound 1) |
| 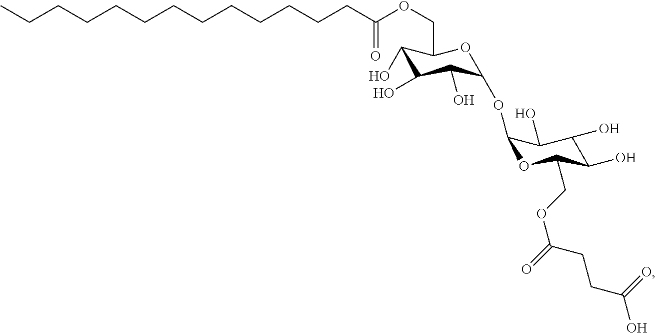 | (Compound 2) |
| 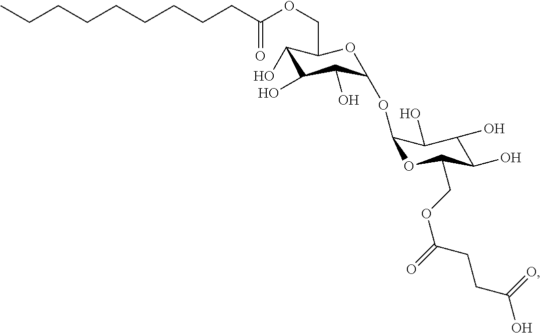 | (Compound 3) |
| 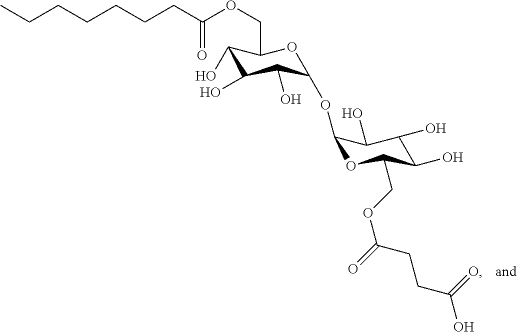 | (Compound 4) |
| 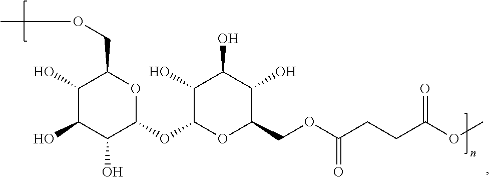 | (Compound 5) |
| with n being an integer between 1-20, and | |
| 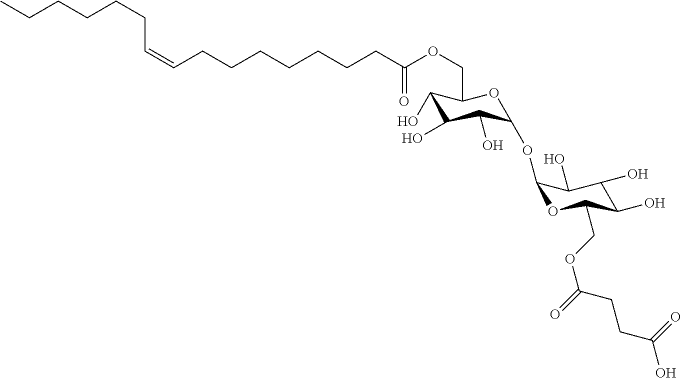 | (Compound 6) |

The present disclosure provides, in some aspects, a composition comprising a compound of formula (I) of the invention. In some preferred aspects, said compound of formula (I) is a surfactant and/or an excipient and/or shows surfactant and/or excipient properties.

The present disclosure further provides, in some aspects, a pharmaceutical composition comprising a compound of formula (I) of the invention. The pharmaceutical composition further comprising a therapeutical effective amount of a pharmaceutical agent.

In some aspects, the pharmaceutical agent is selected from the group comprising a chemical compound, a peptide, a lipid, an oligonucleotide, a cell exosome, and a combination of one or more thereof.

Examples of peptides comprise a peptide a selected from the group comprising an antibody, an antigen binding fragment, and a combination thereof.

As used herein, an “antibody” or “antigen-binding protein or polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

Antibodies may be from any animal origin including birds and mammals. Preferably, the antibodies are human, murine, donkey, rabbit, goat, guinea pig, camel, llama, horse, or chicken antibodies. In another aspect, the antibody or antigen-binding protein is a humanized antibody or a humanized antigen-binding protein.

Examples of oligonucleotides comprise a deoxyribonucleic acid (e.g. DNA, cDNA, . . . ) or ribonucleotide acid (e.g. RNA, miRNA, siRNA, piRNA, hnRNA, snRNA, sgRNA, esiRNA, shRNA, antisense oligonucleotide, lncRNA . . . ) polymer or a combination of deoxyribonucleotide and ribonucleotide (e.g. DNA/RNA) polymer. The oligonucleotide of the invention is in linear or circular conformation, and in either single—or double—stranded form. These terms are not to be construed as limiting with respect to the length of a polymer and can encompass known analogues of natural nucleotides, as well as nucleotides that are chemically modified in the base, sugar and/or phosphate moieties. In general, an analogue of a particular nucleotide has the same base-pairing specificity; i.e., an analogue of A will base-pair with T.

The term “cell exosomes” as used herein refers to a subset of extracellular vehicles (EVs) released by prokaryotes and eukaryotes. Exosomes usually present a size range of ˜40 to 160 nm (average ˜100 nm) in diameter with an endosomal origin. Depending on the cell of origin, exosomes, can contain many constituents of a cell, including DNA, RNA, lipids, metabolites, and cytosolic and cell-surface proteins.

In one aspect, the pharmaceutical composition of the invention further comprises one or more pharmaceutically acceptable carrier and/or diluent.

“Pharmaceutically acceptable carrier or diluent” means a carrier or diluent that is useful in preparing a pharmaceutical composition that is generally safe, non-toxic, and desirable, and includes carriers or diluents that are acceptable for human pharmaceutical use.

Such pharmaceutically acceptable carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions.

The pharmaceutical compositions may further contain one or more pharmaceutically acceptable salts such as, for example, a mineral acid salt such as a hydrochloride, a hydrobromide, a phosphate, a sulfate, etc.; and the salts of organic acids such as acetates, propionates, malonates, benzoates, etc. Additionally, auxiliary substances, such as wetting or emulsifying agents, pH buffering substances, gels or gelling materials, flavorings, colorants, microspheres, polymers, suspension agents, etc. may also be present herein. In addition, one or more other conventional pharmaceutical ingredients, such as preservatives, humectants, suspending agents, other surfactants, antioxidants, anticaking agents, fillers, chelating agents, coating agents, chemical stabilizers, etc. may also be present, especially if the dosage form is a reconstitutable form. Suitable exemplary ingredients include macrocrystalline cellulose, carboxymethyf cellulose sodium, polysorbate 80, phenyletbyl alcohol, chiorobutanol, potassium sorbate, sorbic acid, sulfur dioxide, propyl gallate, the parabens, ethyl vanillin, glycerin, phenol, parachlorophenol, gelatin, albumin and a combination thereof. A thorough discussion of pharmaceutically acceptable excipients is available in REMINGTON'S PHARMACEUTICAL SCIENCES (Mack Pub. Co., N.J. 1991) which is incorporated by reference herein.

The pharmaceutical composition of the invention can be administered orally, buccally, parenterally, nasally, topically or rectally. Preferably, the pharmaceutical composition is administered nasally or orally, more preferably by inhalation.

Preferably also, the pharmaceutical composition of the invention is for use in the treatment of a disease. Any disease is envisioned in the present disclosure.

In one aspect, the pharmaceutical composition of the invention is a vaccine. Vaccines typically contain attenuated, inactivated or dead organisms or purified products derived from them (e.g. antigen). Other examples of vaccines comprise nucleic acid vaccines containing antigens encoded by either DNA or RNA (e.g. mRNA). Said nucleic acid vaccines are usually delivered using viral vectors (such as adenoviruses) or non-viral delivery systems (e.g., electroporation or lipid nanoparticles).

The present disclosure provides, in some aspects, methods of treatment and/or prevention of a disease, said methods comprising administering, to a subject in need thereof, a therapeutically effective amount of a compound of formula (I) of the invention or a pharmaceutical composition of the invention.

The term “therapeutically effective amount” as used herein means an amount a compound of formula (I) high enough to significantly positively modify the symptoms and/or condition to be treated, but low enough to avoid serious side effects (at a reasonable risk/benefit ratio), within the scope of sound medical judgment. The therapeutically effective amount of a compound of formula (I) is selected in accordance with a variety of factors including type, species, age, weight, sex and medical condition of the patient; the severity of the condition to be treated; the route of administration; the renal and hepatic function of the patient. A physician of ordinary skill in the art can readily determine and prescribe the effective amount of the compound of formula (I) required to prevent, counter or arrest the progress of the disease.

As used herein, the term “treatment” or “treating” means any administration of a composition, pharmaceutical composition, therapeutic agent, compound of formula (I), etc., of the disclosure to a subject for the purpose of:
(i) inhibiting the disease, that is, arresting the development of clinical symptoms; and/or
(ii) relieving the disease, that is, causing the regression of clinical symptoms.

As used herein, the term “prevention” or “preventing” means any administration of a composition, pharmaceutical composition, therapeutic agent, compound of formula (I), etc., of the disclosure to a subject for the purpose of preventing the disease, that is, causing the clinical symptoms of the disease not to develop.

The present disclosure also provides a method for preparing a compound of formula (I). The method is described in the example and comprises
a sequential and selective functionalization of the two primary alcohols of trehalose while maintaining the other hydroxyl moieties unreacted,
allowing the conjugation first to a fatty acid chain and then to a carboxylated side chain wherein the carboxylic functionality is introduced in the form of a benzylic ester which is removed at the last step of the procedure.

The present disclosure further provides a method for preparing a polymer of a compound of formula (I), the method comprising autocondensating an activated trehalose derivative in the presence of a coupling agent. Examples of a coupling agent are selected from the group comprising a member of the Carbodiimine familly (such as, e.g. DCC, EDCC, DIC), a member of the Benotriazole family (such as e.g. TBTU, TATU, PyBOP), and a member of the urea family (such as, e.g., Carbodiimidazole (CDI)).

### EXAMPLES

### Example 1

### Synthesis of Succinyl-Trehalose-Fatty Acid (Compound 1 to 4)

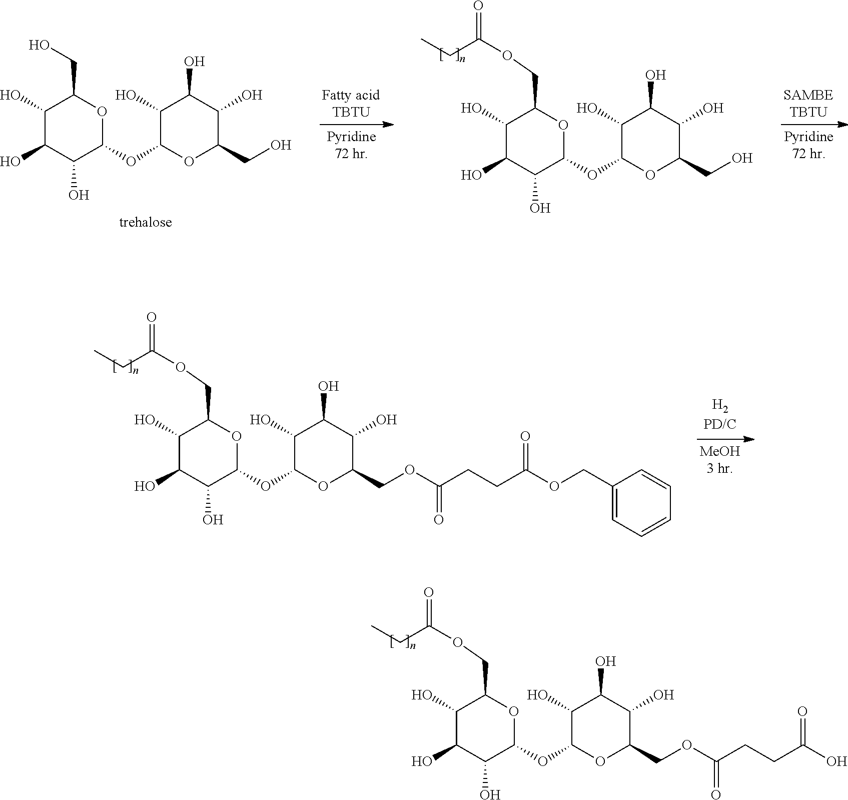

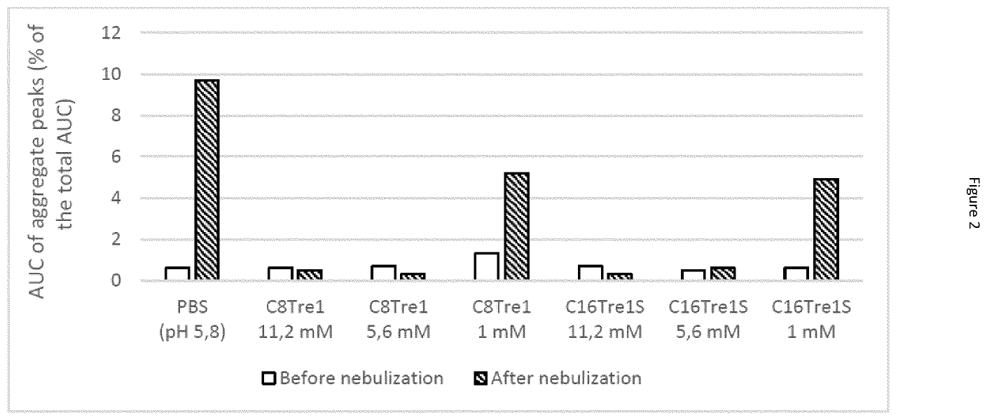

FIG. 2: Overall synthesis of C8TreS

### Synthesis of Trehalose-Fatty Acid:

In a flame-dried round-bottomed flask (20 mL) equipped with a magnetic stir bar, fatty acid (1.1 eq.) and TBTU (0-benzotriazol-1-yl-N,N,N′,N′-tetramethyluronium tetrafluoroborate) (1.1 eq.) were dissolved in anhydrous pyridine (6 mL). Trehalose (D-trehalose) (1 eq.) was then poured into the reaction mixture, and stirring was continued at the r.t for 72 h under argon atmosphere. Pyridine was then removed under vacuum, and the resulting residue was purified using flash column chromatography using a solvent gradient of 5-20% methanol in EtOAc-DCM (1:1). yielding the desired surfactant (yield 51%).

Synthesis of Trehalose-Fatty Acid Comprising Succinyl Functionality with Protecting Group:

In a flame-dried round-bottomed flask equipped with a magnetic stir bar, Succinic acid monobenzyl ester (1.1 eq.) and TBTU (1.1 eq.) were dissolved in anhydrous pyridine (2 mL/0.1 g of trehalose derivative). Trehalose-fatty acid (1 eq.) was then poured into the reaction mixture, and stirring was continued at r.t. for (72 h) under argon atmosphere. Pyridine was then removed under vacuum, and the resulting residue was purified by flash column chromatography using a solvent gradient of 5-20% methanol in EtOAc-DCM(1:1) yielding the desired surfactant (yield 30-40%).

### Synthesis of Succinyl-Trehalose-Fatty Acid:

The fatty acid-trehalose-succinate monobenzyl (1 eq.) was dissolved in MeOH (5 mL) in a 3 necks flask and the system atmosphere was replaced with argon before adding 10% Pd/C. Argon was removed from the system and the reaction was back flushed with hydrogen (3 times). The reaction was vigorously stirred at r.t for 2-3h (monitored by TLC). After completion of the reaction, the solution was filtered through a Celite pad and the filter cake was washed with MeOH (10 mL). The filtrate was concentrated under reduced pressure to yield the desired compound (95%).

### Synthesis of Polymers of Trehalose (Compound 5)

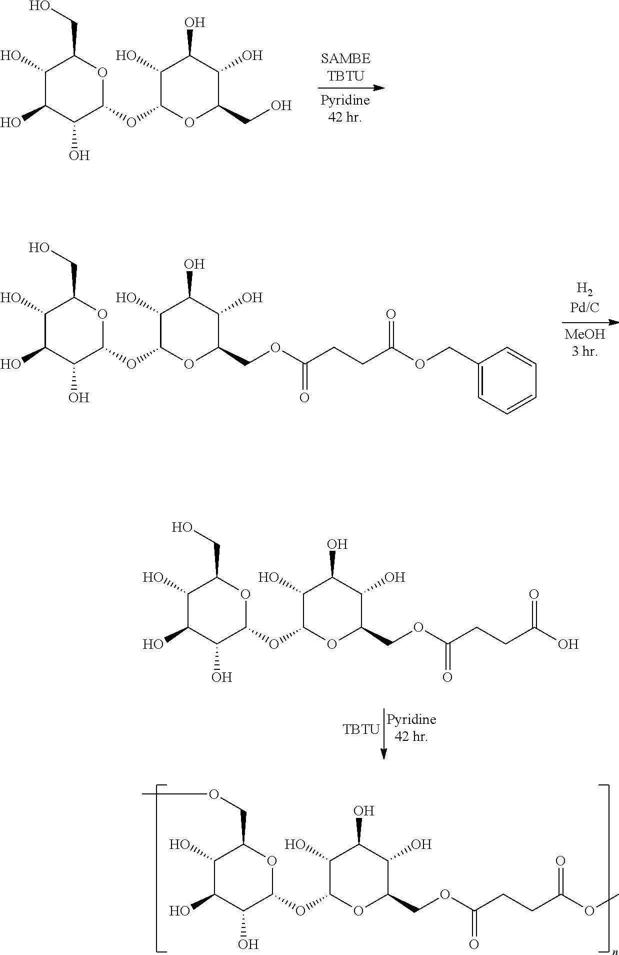

Synthesis of Trehalose Comprising Succinic Functionality with Protecting Group

In a flame-dried round-bottomed flask equipped with a magnetic stir bar, succinic acid monobenzyl ester (SAMBE), as described in Ballard, T. E.; Richards, J. J.; Wolfe, A. L.; Melander, C. Synthesis and Antibiofilm Activity of a Second-Generation Reverse-Amide Oroidin Library: A Structure-Activity Relationship Study. Chemistry—A European Journal 2008, 14, 10745-10761, (1.1 eq.) and 2-(1H-Benzotriazole-1-yl)-1,1,3,3-tetramethylaminium tetrafluoroborate (TBTU) (1.1 eq.) were dissolved in anhydrous pyridine (2 mL/0.1 g of trehalose). Trehalose (1 eq.) was then poured into the reaction mixture and stirred at r.t. for 42 h under argon atmosphere. Pyridine was then removed under vacuum, and the resulting residue was purified by flash column chromatography using a solvent gradient of 9-20% methanol in EtOAc-DCM(1:1). yielding the desired surfactant (yield 37%).

### Synthesis of Trehalose Comprising Succinic Functionality

The trehalose-succinate acid monobenzyl was dissolved in MeOH (1.5 mL/0.1 g of trehalose derivative) in a 3 necks flask and the system atmosphere was replaced with argon before adding 10% Pd/C (cat. ˜10% molar). Argon was removed from the system and the reaction was back flushed with hydrogen (3 times). The reaction was vigorously stirred at r.t for 2-3 hr (monitored by TLC). After completion of the reaction, the solution was filtered through a Celite pad, and the filter cake was washed with MeOH (2× reaction volume). The filtrate was concentrated under reduced pressure to yield the desired compound (83%).

Synthesis of Protected Trehalose Polymer with n=2-5

In a flame-dried round-bottomed flask equipped with a magnetic stir bar, trehalose-succinate acid (1 eq.) and TBTU (1 to 4 eq.) were dissolved in anhydrous pyridine (2 mL/0.1 g of trehalose derivative). The reaction mixture was stirred at r.t. for 42 h under argon atmosphere. Pyridine was then removed under vacuum, and the resulting residue was purified by dialysis (100-500 MWCO).

### Synthesis of Succinyl-Trehalose-Unsaturated Fatty Acid

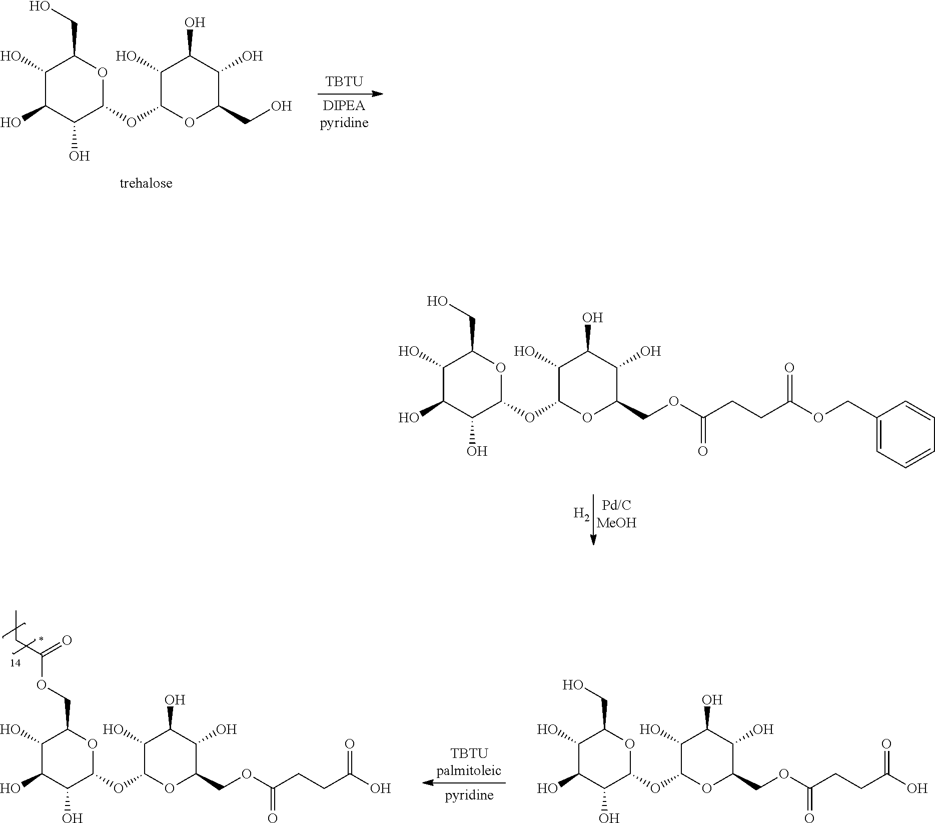

### Synthesis of Succinyl-Trehalose:

The intermediate (1 eq.) was dissolved in MeOH (1.3 mL/0.1 g of intermediate) in a 3 necks flask and the solution was placed under argon atmosphere before adding 10% Pd/C (0.1 eq.). Argon was removed from the reactor and the reaction mixture was back flushed with hydrogen (1 atm, 3 times). The reaction was vigorously stirred at r.t for 4 hr (monitored by TLC) under hydrogen atmosphere. After completion of the reaction, the solution was filtered through a Celite pad and the filter cake was washed with MeOH. The filtrate was concentrated under reduced pressure yield succinyl trehalose as white solid (quant.).

### Synthesis of Succinyl-Trehalose-Unsaturated Fatty Acid:

In a flame-dried round-bottomed flask equipped with a magnetic stir bar, palmitoleic acid (1.2 eq.) and TBTU (1.4 eq.) were dissolved in anhydrous pyridine (3 mL/0.1 g of palmitoleic acid). The reaction mixture was stirred at r.t. for 1 hr before the addition of TreSuc (1 eq.). Stirring was continued at r.t. for 96 hr under argon atmosphere. Pyridine was then removed under vacuum, and the resulting residue was purified by reversed phase flash chromatography (H₂O:MeCN 4:6→2:8). The fractions corresponding to the product were collected, concentrated in vacuo and lyophilized to yield the desired compound as white solids (yield 15%).

Formulation preparation procedure Trehalose surfactant CnTreS solution was prepared by dissolution of 5.6 mmol trehalose surfactants in 1 L of a commercially available solution of PBS buffer 1×. Then, 6.3 mg/L antibody solution (provided in PBS (pH 6.5-6.7) with 100 mM L-arginine) was diluted in the trehalose surfactant solution to reach 1 mg/mL concentration.

### Nebulization Procedure

500 uL to 8 mL of antibody formulation was nebulized with a Briutcare mesh nebulizer (NEB-001). The flow rate was set up at 0.4 mL/min and the aerosol solution was retrieved in a 50 mL falcon tube.

### Freeze-Drying Procedure

500 uL of 1 mg/mL antibody formulations were placed in a vial prior to deep-freeze in liquid nitrogen. The frozen samples were placed directly in the freeze-dryer. After 24 hr, the lyophilized samples were stored in a fridge at 4° C. for 7 or 28 days. The sample were then reconstituted with addition of MilliQ® water before assessing their stability by DLS and cell binding assays.

### Stability Assessment

The stability of the antibody solution dissolved in trehalose surfactant solution was studied over a period of 10 days at 25° C. Antibody aggregations and conformations were measured by mean of dynamic light scattering DLS using a nano-ZS (Malvern Panalytical) and nano-DSF apparatus (NanoTemper Technology) respectively while Fab ability to interact with its antigen functionality was assessed by ELISA assay developed for the study.

### Cell Binding Assays

The functional stability was evaluated by studying its ability to bind antigen on cell membranes. Wild type HEK-cells and SARS-CoV-2 spike (D614)-expressing HEK-cells were placed into culture in RPMI medium, with 10% of fetal bovine serum, 100 U/ml penicillin, and 100 μg/ml streptomycin. Both cell lines were incubated with 50 μL of 0.5 pg/mL antibody formulations for 30 min at 4° C. The cells were washed in PBS, and alexa Fluor 488-conjugated mouse anti-human Fc IgG diluted at 1/500 in PBS was added prior incubation for 15 min in the dark at 4° C. The cells were washed, and fluorescence activated cell were analysed by FACS using CANTO II (BD Bioscience, USA).

### Colloidal Stability Assessment

The aggregation of antibody was assessed by dynamic light scattering (DLS) operating at 25° C. The measurements were done at different timepoints during storage at 25° C. and right after nebulization.

The mean hydrodynamic diameter by intensities of the monomer was distributed at d=10 nm while aggregates was distributed at a d >10 nm.

### Cytotoxicity Assays

The cytotoxicity of trehalose surfactants was assessed on NIH-3T3 cell line. Trehalose surfactants solutions ranging from 0.0112 to 11.2 mM were prepared by dilution of the stock solution into the cell culture medium (DMEM with 10% of fetal bovine serum completed with 2 mM glutamine and 1% penicillin/streptomicyn). The cells were incubated with diluted surfactants for 48 h at 37° C. and a solution of MTS/PMS (MTS CellTiter96 AQueous assay) was added following supplier instructions. Cells were then incubated for 3 h at 37° C. and absorbance was measured at 490 nm using a MultiSkan microplate reader (Thermo Fisher Scientific, USA). Cell viability was plotted against the surfactant concentration, and the median cytotoxic dose (CD50) was calculated

### Example 2

### Synthesis

Trehalose surfactants are produced from biocompatible, non-toxic, and cheap raw material, (saturated or unsaturated fatty acid, trehalose and succinic acid) available from green sources. They can be synthetized by desymetrization strategy from unprotected trehalose. They can be easily degraded and metabolized by the human body by enzymatic and hydrolytic cleavage of the ester bonds. In one embodiment, the excipients comprise a succinate functionality to enhance interaction between the biomolecule and the excipients via electrostatic interactions. In another aspect, the excipients comprise 2 units of trehalose to increase the hydrophilicity portion of the surfactants, to improve the solubility of the biomolecule/surfactant complex in an aqueous solution and to enhance the biomolecules stability through the formation of the hydrogen bonding.

### On Shelf Stability

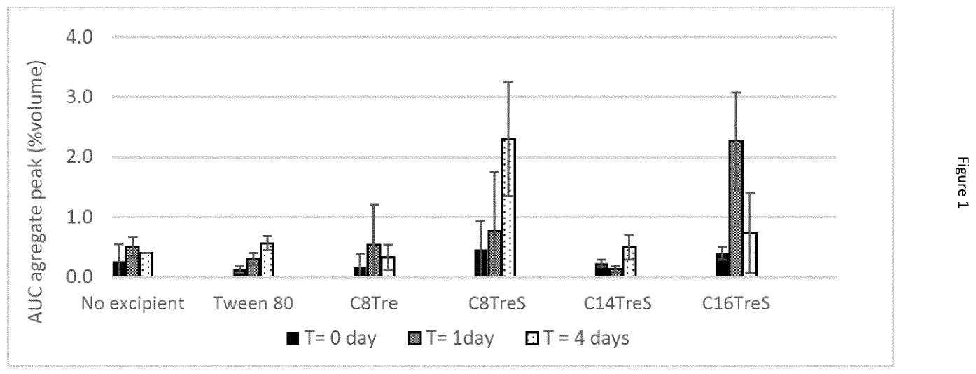

Inventors have shown that trehalose surfactants enhance the biomolecule solubility with the formation of drug-surfactant colloidal complexes while improving their shelf-life stability. The shelf-life stability was monitored over 4 days by evaluated aggregates formation by DLS (FIG. 1). IgG at 1 mg/mL in PBS showed aggregations after 4 days with and without excipients.

### Stability During Nebulization

The use of trehalose surfactants in the antibody formulations decrease aggregations and conformational changes during nebulization. The stability of the antibody after nebulization was assessed by DLS and nano-DSF to evaluate aggregations formation and conformational changes respectively after nebulization on freshly prepared formulations. As shown in FIG. 2, nebulization in PBS only presented 9.7% aggregates of the total AUC. It seems that nebulization dramatically accelerate aggregation, in comparison to the on-shelf aggregation rate observed during storage at 25° C. (3.5% in PBS only after 10 days).

For C8Tre1 or C16Tre1S at 1 mM, aggregation was observed (around 5% of the total volume-weighted AUC). Aggregates were avoided when formulations contained C8Tre1 or C16Tre1S at a minimum concentration of 5.6 mM. The data are in good agreement with the shelf-life stability studies.

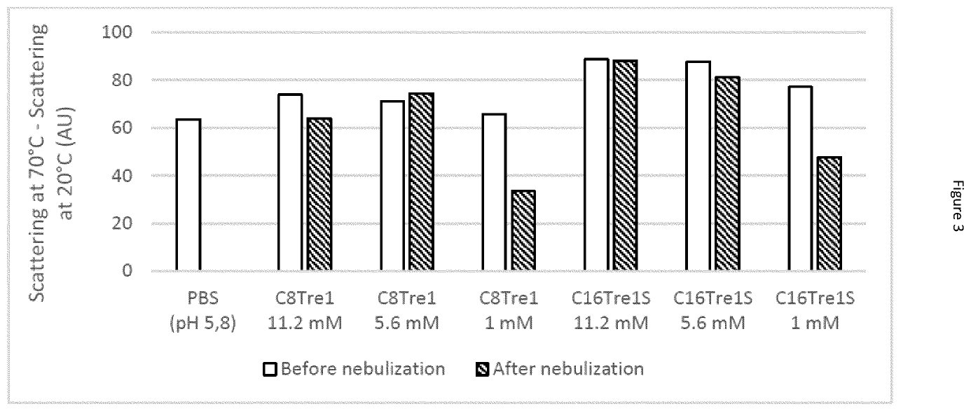

The colloidal stability of the antibody after nebulization was also assessed by the Nano-DSF. The difference between the scattering value at 70° C. (highest level of aggregation) and at 20° C. (lowest level of aggregation) was calculated for each sample, before and after nebulization (FIG. 3).

The results showed that for most of the samples, similar results were obtained before and after nebulization. However, trehalose surfactants at 1 mM showed aggregates probably generated by the nebulization. The data are in good agreement with the DLS experiments showing aggregation at the same concentration. C8Tre1 and C16Tre1S prevented antibody aggregation when used at a minimal concentration of 5.6 mM. No changes before and after nebulization suggested that the same proportion of aggregates are presents before and after heating with the nano-DESF equipment.

### Stability During Freeze-Drying:

The addition of 5.6 mM C16Tre1S and C8Tre1S trehalose surfactants as cryoprotectant was assessed on 1 mg/mL antibody formulations. The samples were first deep-frozen in liquid nitrogen before drying for 24 hr. The lyophilized samples were stored in a fridge for 0, 7 or 28 days before reconstitution with addition of MilliQ® water. The conformation and functionality of antibody after the process were assessed by DLS and cell binding assays respectively. The results were compared to antibody formulated in PBS only and formulated with standard cryoprotectants such as trehalose (5 wt % and 5.6 mM) and Tween 80 (5.6 mM).

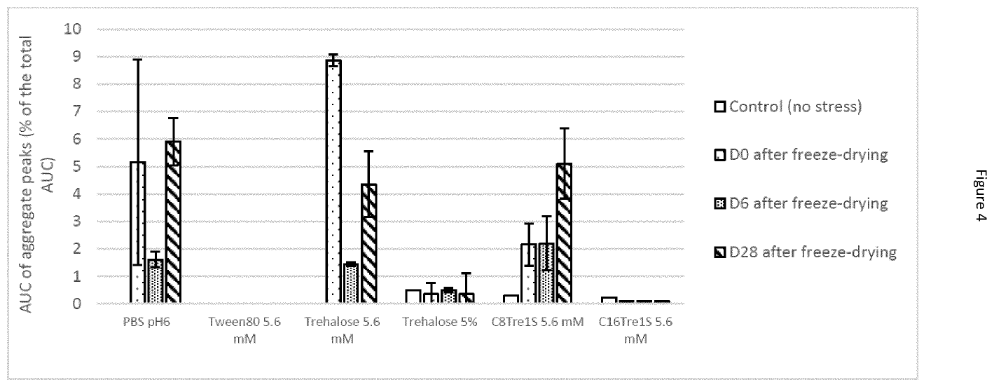

The DLS results (FIG. 4) showed that no aggregations were observed for the formulation containing C16Tre1S and the lyophilized antibody remained stable when stored at 4° C. for 28 days. Comparable results were obtained with the two standard cryoprotectants (Tween 80 at 5.6 mM and Trehalose at 5 wt %). However, aggregations were observed when low amount of Trehalose was used (5.6 mM) as for samples containing PBS only. For samples containing C8Tre1S, aggregation after freeze-drying was observed and the amount of aggregates increased with the time of storage, suggesting that C8Tre1S was cryoprotective but do not stabilized antibody during storage.

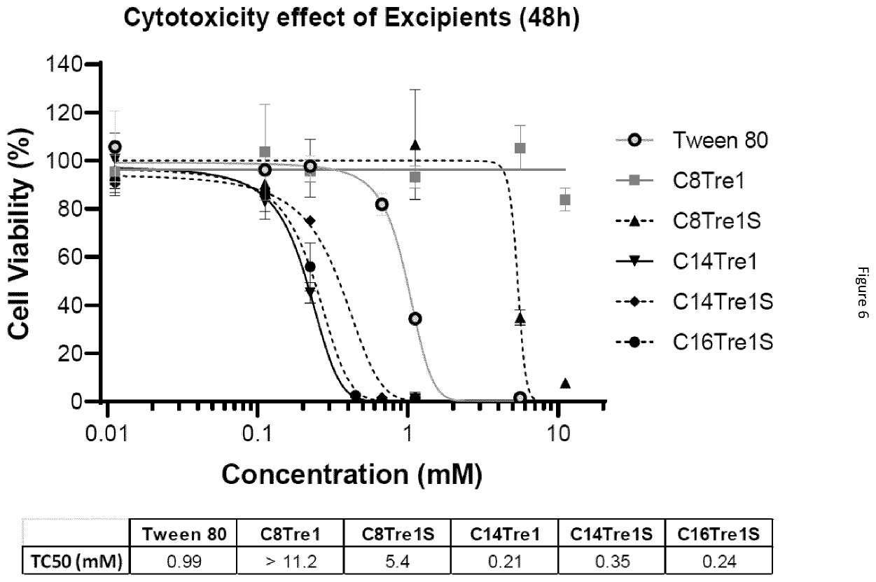

Then, the ability of the antibody to bind to its antigen after lyophilization was assessed in vitro using SARS-CoV-2 spike-expressing HEK293 cells (FIG. 6). After antibodies treatment, cells were labelled by Alexafluor 488. High fluorescence signals were observed for all HEK293 cells expressing spike proteins when antibodies were formulated with C16TreS. Comparable results were obtained with the standard cryoprotectant Tween 80 at the same concentration. However, significant decrease of antibody binding was observed for formulation with Trehalose (5 wt % and 5.6 mM), C8TreS (5.6 mM) and no surfactants. Then, the reconstituted antibody formulations containing Tween 80 and C16TreS was submitted to nebulization challenge and antibodies were able to binds Spike protein at the same level as before nebulization. These results suggest that C16Tre1S trehalose surfactants stabilize antibody during freeze-drying, storage over 28 days when lyophilized and nebulization process at a comparative extent to the standard cryopreservative Tween 80.

### Cytotoxity

The cytotoxicity of all the surfactants used in the study was assessed on NIH-3T3 cells after 48h with a MTS cell viability test. C8Tre1 showed a CD50 of 5.4 mM, versus 0.30 mM, 0.84 mM and 0.24 mM for C14Tre1, C14Tre1S, or C16Tre1S respectively. It seems that the longer the alkyl chain, the higher the cytotoxicity.

Also, succinylation of the trehalose appeared to increase the toxicity, as CD50 was of 5.4 mM for C8Tre1S, versus more than 11.2 mM for C8Tre1. Anionic surfactants are often considered as more toxic than non-ionic surfactants.

1. A compound of formula I 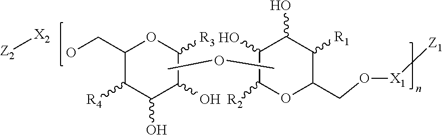
or a salt, stereoisomer, polymorph or mixture of stereoisomers thereof,
wherein:
R₁ is selected from —OH and an alkyl, optionally substituted with OH; or R₁ is linked to R₃ via an oxygen;
R₂ is selected from —OH and an alkyl, optionally substituted with OH or R₂ is linked to R₃ via an oxygen;
R₃ is selected from —OH and an alkyl, optionally substituted with OH; or R₃ is linked to R₁ or R₂ via an oxygen;
R₄is selected from —OH and an alkyl, optionally substituted with —OH
X₁ is independently selected from, —(C═O) and —(C═O)-A-(C═O), wherein A is an alkyl, an alkenyl, an alkynyl, or PEG;
X₂ is selected from H and —(C═O);
Z₁ is —OH
and,
when X₂ is —(C═O), then Z₂ is selected from alkyl, alkenyl, alkynyl, PEG, succinyl, —NH alkyl, —NH alkenyl, —NH alkynyl, NH-PEG, —S alkyl, —S alkynyl, —O alkyl, —O alkenyl, and —O alkynyl; and
when X₂ is H, then Z₂ is absent;
and n is an integer 1-20.

2. The compound of claim 1, wherein
R₁ is —OH
R₂ is linked to R₃ via an oxygen-;
R₄ is OH
XI is —(C═O)—CH2-CH2-(C═O);
Z₁ is —OH
X₂ is —(C═O); and
Z₂ is an alkyl or, alkenyl.

3. The compound of claim 1, wherein said compound is selected form the group consisting of;

| | |
|---|---|
| 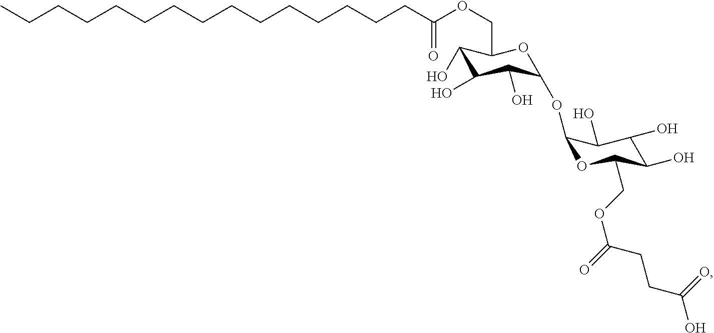 | (Compound 1) |
| 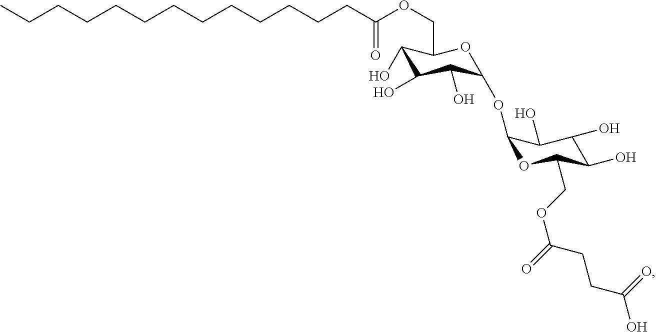 | (Compound 2) |
| 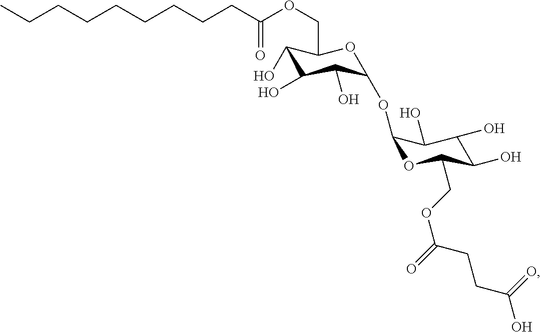 | (Compound 3) |
| 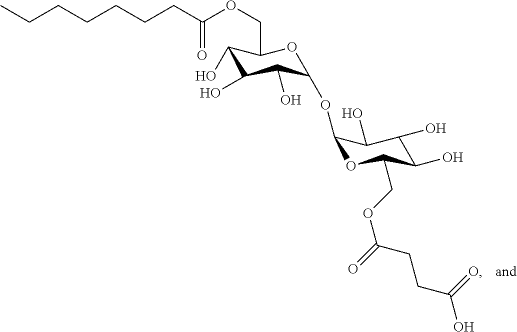 | (Compound 4) |
| 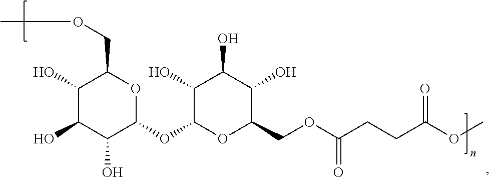 | (Compound 5) |
| with n being an integer 1-20, and | |
| 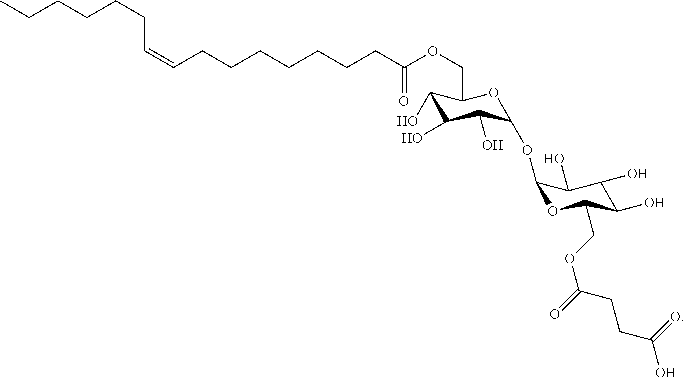 | (Compound 6) |

4. A composition comprising a compound of formula I according to claim 1.

5. The composition of claim 4, wherein said compound of formula I is a surfactant or an excipient.

6. A pharmaceutical composition comprising a compound of formula I according to claim 1.

7. The pharmaceutical composition of claim 6, further comprising a therapeutically effective amount of a pharmaceutical agent.

8. The pharmaceutical composition of claim 7, wherein the pharmaceutical agent is selected from the group consisting of a chemical compound, a peptide, a lipid, an oligonucleotide, a cell exosome, and a combination of one or more thereof.

9. The pharmaceutical composition of claim 8, wherein the peptide is selected from the group consisting of an antibody, an antigen binding protein, and a combination thereof.

10. The pharmaceutical composition of claim 8, wherein the oligonucleotide is selected from the group consisting of a deoxyribonucleic acid, a ribonucleic acid, and a combination thereof.

11. (canceled)

12. The pharmaceutical composition of claim 6, wherein said pharmaceutical composition is in a dosage form to be administered orally, buccally, parenterally, nasally, topically or rectally.

13. The pharmaceutical composition of claim 12, wherein said pharmaceutical composition is in a dosage form to be administered nasally or orally, by inhalation.

14. The pharmaceutical composition of claim 6, wherein the pharmaceutical composition is a vaccine.

15. A method for preparing a compound of formula I of claim 1, the method comprising;
a sequential and selective functionalization of the two primary alcohols of trehalose while maintaining the other hydroxyl moieties unreacted,
allowing the conjugation first to a fatty acid chain and then to a carboxylated side chain, wherein the carboxylic functionality is introduced in the form of a benzylic ester which is removed at the last step of the procedure.

16. A method for preparing a polymer of a compound of formula I of claim 1, the method comprising auto-condensating an activated trehalose derivative in the presence of a coupling agent.

17. A method of treatment and/or prevention of a disease, said method comprising administering, to a subject in need thereof, a therapeutically effective amount of a compound of formula I of claim 1 or a pharmaceutical composition comprising the compound of formula I.

18. The method of claim 17, wherein the compound of formula I or the pharmaceutical composition comprising the compound of formula I is administered orally, buccally, parenterally, nasally, topically or rectally.

19. The method of claim 18, wherein the compound of formula I or the pharmaceutical composition comprising the compound of formula I is administered nasally or orally, by inhalation.
